(19) United States
(12) Patent Application Publication
(10) Pub. No.: US 2009/0017449 A1
(43) Pub. Date: Jan. 15, 2009

(54) COMPOUNDS AND METHODS FOR ASSAYING FUSION OF AN INDIVIDUAL, ENVELOPED VIRUS WITH TARGET MEMBRANE

(75) Inventors: Antoine M. Van Oijen, Cambridge, MA (US); Daniel FLOYD, Allston, MA (US)

Correspondence Address: BANNER & WITCOFF, LTD., 28 STATE STREET, 28th FLOOR, BOSTON, MA, 02109-9601, US

(73) Assignee: President and Fellows of Harvard College, Cambridge, MA (US)

(21) Appl. No.: 12/142,965

(22) Filed: Jun. 20, 2008

Related U.S. Application Data
(60) Provisional application No. 60/945,134, filed on Jun. 20, 2007.

(51) Int. Cl. C12Q 1/70

(52) U.S. Cl. 435/5

(57) ABSTRACT

Compositions and methods for monitoring viral fusion are provided. Methods of labelling virions are also provided. A novel, detectable label is provided. A mobile lipid bilayer is also provided.

### RELATED U.S. APPLICATION

This application claims priority to U.S. Provisional Patent Application No. 60/945,134, filed on Jun. 20, 2007, hereby incorporated herein by reference in its entirety for all purposes.

### STATEMENT OF GOVERNMENT INTERESTS

This application was funded in part by grant number 1 R21 AI072346 from the National Institutes of Health/National Institutes of Allergy and Infectious Disease. The Government has certain rights in the invention.

### FIELD

The present invention relates to compounds and methods for assaying viral mediated membrane fusion.

### BACKGROUND

Before a virus can infect an organism, it must introduce its genomic content into the target cell. For enveloped viruses (such as, for example, influenza and HIV), this first key step requires the fusion of the lipid bilayer that surrounds the viral particle with a cell membrane. Existing techniques to characterize the kinetics of fusion and to study the effect of small-molecule inhibitors on this process rely on the expression of large numbers of fusion proteins on a cell surface and its fusion with a second target cell. The technical complexity of expressing fusion proteins on a cell surface and the non-physiological context that such a method produces places severe restrictions on the application of these techniques to the development of drugs that effectively inhibit viral fusion. Moreover, the large number of fusion events that mediate cell-cell fusion in such assays result in an ensemble averaging that causes details of the different steps in the fusion process to be invisible.

Enveloped viruses (including, for example, viruses such as influenza, dengue, West Nile, yellow fever, hepatitis B, HIV, HSV and the like) have a lipid bilayer enclosing their genomic content. A critical event in the infection of a target cell by an enveloped virus is the fusion of the viral envelope with a lipid membrane of the target cell. This important step allows the content of a viral particle to be introduced into the target cell (Harrison (2005) Adv. Virus Res. 64:231; Eckert and Kim (2001) Ann. Rev. Biochem. 70:777; Tamm et al. (2003) Curr. Opin. Struct. Biol. 13:453; Sollner (2004) Curr. Opin. Cell Biol. 16:429; Cohen and Melikyan (2004) J. Membr. Biol. 199:1). The kinetically unfavorable membrane fusion process is mediated by specialized fusion proteins that reside on the surface of the viral particle. The overall pathway is well established and proceeds through a series of intermediates: local membrane deformation and contact; creation of hemifused membranes; formation of a fusion pore and pore enlargement (FIG. 1).

The first step of the fusion process involves deformation of the lipid bilayers by the fusion proteins to bring the initially separated viral and target membranes into close proximity (FIG. 1). Subsequently, the two proximal lipid leaflets merge and form a hemifusion stalk. Fusion is then thought to proceed to the formation of an extended hemifusion intermediate that allows lipids between the viral and target membranes to exchange. Finally, a fusion pore is formed that enables the aqueous content to freely move between the viral and target compartments.

The fusion process is a biochemical pathway that contains many intermediates, a wide range of transition rates, and possibly routes leading to both successful fusion and abortive, dead-end states. The combined results of many fusion studies have greatly enhanced our understanding of this complex pathway, but the small size of a viral particle (approximately 50-100 nm in diameter) limits the experimental techniques available to observe viral-mediated membrane fusion. By expressing fusion protein on the surfaces of cells, large areas of fusogenic membrane can be formed and their fusion with a target membrane can be more easily studied. However, the averaging over many fusion events makes a straightforward kinetic and quantitative analysis difficult. Furthermore, these ensemble-averaged experiments only allow the observation of fusion intermediates that are sufficiently long-lived to build up to an appreciable portion of the population.

### SUMMARY

The present invention is based in part on the discovery of novel methods for observing and characterizing real-time fusion of individual and/or intact enveloped viruses with a target membrane. The methods and compositions described herein utilize a fundamentally novel strategy for assaying viral fusion, i.e., reconstituting viral fusion in vitro with only the bare minimum of molecular components and monitoring the dynamics of the fusion process at the single-particle level. Observation of fusion at the single-event level provides at least two important advantages over methods known in the art: 1) the absence of population averaging; and 2) the absence of temporal dephasing. Monitoring a single fusion event will enable one of skill in the art the ability to discern whether different subpopulations exist with different properties. For example, one of skill in the art will be able to determine whether a pair of intermediates occurs successively in all fusion events, or whether one intermediate appears only in one subpopulation of events and the other state in yet another set of events.

The stochastic nature of biochemical processes causes a population of reactions to lose its synchronicity quickly after initiation of the reaction. The ensemble dephasing makes it impossible to observe any short-lived intermediates that occur during fusion. The novel methods and compositions described herein further allow for the study of viral fusion with significantly reduced volumes of reagents. Only a few dozen individual fusion events are required to build up a reliable data set that accurately reflects the important kinetic parameters of fusion. By combining an in vitro fusion assay as described herein with high-magnification microscopy, fusion on sample sizes of only 50×50 μm² was observed. The use of microfluidics (e.g., silicon based organic polymer fluidics, e.g., polydimethylsiloxane) will enable the observation of viral fusion using sub-nanoliter volumes of reagents. This development will aid in the screening of large small-molecule libraries to identify compounds effective in inhibiting viral fusion.

Accordingly, in certain exemplary embodiments, a method of monitoring virus-mediated fusion is provided. The method includes providing a labelled, enveloped particle containing one or more viral proteins, wherein the particle has a detectably labelled envelope and/or a detectably labelled internal region, providing a target membrane, contacting the target membrane with the labelled, enveloped particle, and monitoring fusion. In certain aspects, the envelope and/or the internal region each have a fluorescent label. In other aspects, the envelope and the internal region each have a different detectable label. In yet other aspects, the envelope contains a lipophilic, detectable label (e.g., Rh110C18) and/or the internal region contains a water soluble, detectable label (e.g., sulforhodamine B). In certain aspects, viral fusion is monitored for a single, enveloped virion, such as an intact virion. In certain aspects, the labelled, enveloped particle containing one or more viral proteins is a virion, a virosome or a virus-like particle.

In certain aspects, hemifusion and/or formation of a fusion pore is monitored by observing an increase or decrease in one or more photophysical properties (e.g., fluorescence intensity, fluorescence lifetime, emission wavelength, absorption wavelength, polarization and the like) of the lipophilic, detectable label or the water soluble, detectable label, respectively. In certain aspects, hemifusion is monitored by observing an instantaneous increase in brightness of the lipophilic, detectable label, and in other aspects, the instantaneous increase in brightness of the lipophilic, detectable label is followed by a decrease in brightness of the of the lipophilic, detectable label. In still other aspects, formation of a fusion pore is monitored by observing a decrease in brightness of the water soluble, detectable label.

In certain aspects, the target membrane can be any combination of a phospholipid bilayer, a liposome, a membrane fragment (e.g., one or more bicelles) and/or an array (e.g., an array of any combination of one or more bilayers (e.g., phospholipid bilayers), liposomes, and/or membrane fragments). In other aspects, the target membrane is attached to a support such as, e.g., a microscope slide, a multi-well plate or a microfluidic support. At least a portion of the support may optionally be coated with a substance that binds phospholipids such as, for example, dextran. In certain aspects, monitoring is performed by microscopy, such as, e.g., fluorescence microscopy.

In certain exemplary embodiments, a method of labelling a virion including providing a virion, providing a water soluble label, and contacting the virion with the water soluble label to generate a labeled virion is provided. In certain aspects, the water soluble label is a fluorescent label such as, e.g., sulforhodamine B.

In other exemplary embodiments, a method of labelling a virion including providing a virion, providing Rh110C18, and contacting the virion with the Rh110C18 to generate a labeled virion is provided.

In certain exemplary embodiments, an intact virion comprising a water soluble, detectable label, wherein the water soluble, detectable label is present inside the virion, is provided.

In certain exemplary embodiments, a method of labelling a virion is provided. The method includes providing a virion, providing Rh110C18, and contacting the virion with the Rh110C18 to generate a labeled virion.

In certain exemplary embodiments, a mobile lipid bilayer is provided. The mobile lipid bilayer includes a glass support, wherein at least a portion of the support is derivatized with dextran, and a lipid bilayer attached to a least a portion of the dextran. In certain aspects, the lipid bilayer further comprises a detectable label. In other aspects, the lipid bilayer is a phospholipid bilayer. In still other aspects, the lipid bilayer further comprises one or more viral receptors.

In certain exemplary embodiments, a detectable label having the structure

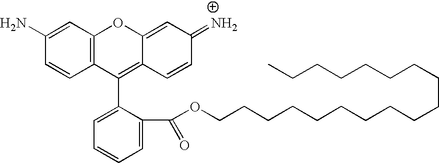

is provided.

In certain exemplary embodiments, a method of monitoring one or more physical conditions inside a virion is provided. The method includes providing a virion having a water soluble, detectable label inside the virion, and observing an increase or decrease in one or more photophysical properties of the water soluble, detectable label. In certain aspects, the one or more photophysical properties are selected from fluorescence intensity, fluorescence lifetime, emission wavelength, absorption wavelength and/or polarization. In other aspects, the one or more physical conditions inside a virion includes a change in pH, such as, for example, a decrease in pH. In still other aspects, the virion is an influenza virion.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The foregoing and other features and advantages of the present invention will be more fully understood from the following detailed description of illustrative embodiments taken in conjunction with the accompanying drawings in which:

FIG. 1 schematically depicts the proposed fusion mechanism for class I viral proteins.

FIG. 2 schematically depicts influenza virus entry. The virus is taken up by endocytosis and transported to an endosome. The acid environment of the endosome initiates fusion of the viral envelope with the endosomal membrane, releasing viral RNA and proteins.

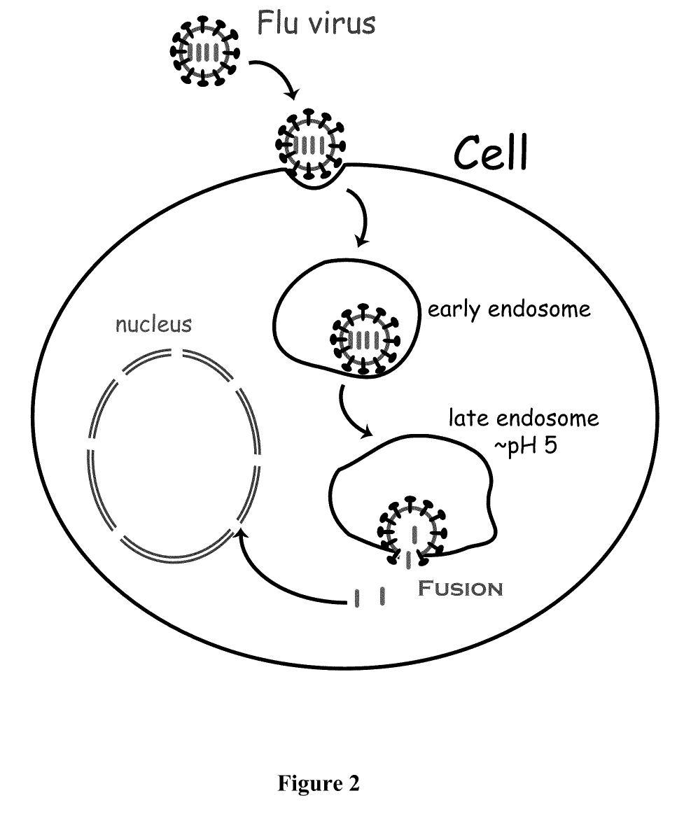

FIGS. 3A-3E graphically depict fusion kinetics under varying pH conditions. A-C) hemifusion (A), pore-formation (B) and hemifusion decay (C) histograms are shown for events recorded at varying acidic pH conditions. D) Kinetic rate constants for transitions between pre-hemifusion intermediates (green squares) and decay of hemifusion to formation of fusion pores (gray circles) plotted as a function of proton concentration. The solid lines are plotted from least-squares fit (y=a+bx) of the rate constants as a function of proton concentration (green line: a=−0.01135, b=8114, R=0.987; gray line: a=0.03522, b=160.6, R=0.635). The lines appear curved in the log-log plot as a result of the non-zero y-intercepts.

FIGS. 4A-4B schematically depict optical design of experiments described herein. A) Fusion of fluorescently labeled viral particles with a planar bilayer can be monitored using evanescent excitation. B) Different dyes are simultaneously excited and detected at multiple wavelengths.

FIGS. 5A-5D depict measurement of membrane fluidity. A) Fluorescence recovery of photobleaching. A circular area is photobleached and B) its recovery measured. C) Single-particle tracking. Individual, fluorescent lipids are imaged and their positions tracked. D) Their mean-square displacement as a function of time provides a diffusion coefficient.

FIGS. 6A-6B depict hemifusion and pore formation of individual influenza viruses with a planar bilayer. A) Fluorescence images before (left) and during (right) fusion of individual viral particles with a target membrane. B) The fluorescence intensity of the red sulforhodamine B (SRB) viral content tracer (upper trace), the green Rh110C18 membrane dye (middle trace), and the fluorescein pH sensor (lower trace) provides the exact time elapsed between pH drop and fusion.

FIGS. 7A-7C depict fusion kinetics of fluorescently labeled influenza virus. A) Time elapsed between pH decrease and hemifusion (green) and pore-formation (red) of individual particles. The presence of intermediate states before hemifusion is clearly visible as a rise and decay in the histograms. Solid lines are best fits to a gamma function with N transitions (N=3 for hemifusion (green); N=4 for pore formation (red)). The dashed line represents a convolution of the N=3 gamma distribution of hemifusion times with the experimentally observed single-exponential transition between hemifusion and pore formation. B) Hemifusion histogram from A) is compared to gamma distribution fits with varying numbers of steps. The inset graph shows the fitting error for fits with one to ten transitions. C) Distribution of lag-times between hemifusion and pore-formation of individual particles. The solid line represents a single-exponential fit with a rate constant of 0.55±0.004 sec⁻¹.

FIG. 8 graphically depicts the detection of proton influx before hemifusion. Fluorescence trajectories of particles containing fluorescein show loss of content signal prior to hemifusion. Without intending to be bound by scientific theory, distortion of the viral envelope prior to hemifusion might allow protons from the acidic exterior to leak inside the virus. Alternatively, without intending to be bound by scientific theory, quenching might result from activation of the M2 proton channel.

FIGS. 9A-9D depict scatter plots of the fusion lag times from FIG. 6A as a function of fluorescent dye intensity from individual virus particles. Hemifusion and pore-formation times are plotted as a function of green (lipid) dye intensity (A and B) and red (content) dye intensity (C and D). The correlation coefficient (R) for each set of lag time and dye intensity is also shown. The low correlation between lag time and dye intensity indicates that incorporation of fluorescent dyes has little effect on fusion kinetics.

### DETAILED DESCRIPTION

The principles of the present invention may be applied with particular advantage to assay different steps of membrane fusion (e.g., hemifusion and/or pore formation and/or complete fusion) between individual virions and a target membrane.

In certain exemplary embodiments, one or more particles containing one or more viral proteins (e.g., viruses) are used in the compositions and/or assays described herein. As used herein, the term virus includes DNA or RNA animal viruses. As used herein, RNA viruses include, but are not limited to, virus families such as picornaviridae (e.g., polioviruses), reoviridae (e.g., rotaviruses), togaviridae (e.g., encephalitis viruses, yellow fever virus, rubella virus), orthomyxoviridae (e.g., influenza viruses), paramyxoviridae (e.g., respiratory syncytial virus, measles virus, mumps virus, parainfluenza virus), rhabdoviridae (e.g., rabies virus), coronaviridae, bunyaviridae, flaviviridae, filoviridae, arenaviridae, bunyaviridae, and retroviridae (e.g., human T-cell lymphotropic viruses (HTLV), human immunodeficiency viruses (HIV)). As used herein, DNA viruses include, but are not limited to, virus families such as papovaviridae (e.g., papilloma viruses), adenoviridae (e.g., adenovirus), herpesviridae (e.g., herpes simplex viruses), and poxviridae (e.g., variola viruses).

In certain exemplary embodiments, one or more enveloped viruses are used in the compositions and/or assays described herein. As used herein, the term “enveloped virus” refers to a virus that contains a membrane that envelopes the virion. As used herein, the term “virion” refers to a virus particle which typically comprises a nucleic acid surrounded by a capsid. The membrane is derived from the outer membrane of an infected host cell or from host cell internal membranes. Proteins (e.g., viral glycoproteins) embedded in the envelope serve to bind to receptor sites on the host cell membrane (e.g., viral attachment proteins). Proteins (e.g., viral fusion proteins) also mediate fusion between the virion and the host cell. Viral attachment proteins and viral fusion proteins may be separate proteins (e.g., H/HN/G proteins and F proteins of paramyxoviruses). Alternatively, a single viral protein can function to bind one or more receptors and mediate membrane fusion (e.g., HA proteins of orthomyxoviruses). Examples of enveloped virus families include, but are not limited to, togaviridae, flaviviridae, bunyaviridae, arenaviridae, coronaviridae, herpesviridae, orthomyxoviridae, paramyxoviridae, poxyiridae, retroviridae and rhabdoviridae.

In certain exemplary embodiments, one or more particles containing one or more viral proteins are used in the compositions and/or assays described herein. Such particles include, for example, virosomes and virus-like particles. As used herein, the term “virosome” refers to vesicles (e.g., vesicles comprising a phospholipid bilayer) that can contain one or more viral proteins (e.g., fusion proteins). Virosomes are typically devoid of genetic material. As used herein, the term “virus-like particle” refers to particles having one or more viral structural proteins (e.g., capsid proteins). Virus-like particles may optionally contain a lipid bilayer and/or viral fusion proteins. Like virosomes, virus-like particles typically lack genetic material. Particles containing one or more viral proteins also include viruses and/or virions.

Viruses, virions, particles containing one or more viral proteins, viral fusion proteins and their receptors are further described in Fields Virology 5^{th} Ed., Knipe et al. (2007) Lippincott, Williams & Wilkins, incorporated herein by reference in its entirety for all purposes.

In certain exemplary embodiments, a target membrane, e.g., a supported lipid bilayer, can be used whose composition can be controlled to mimic a cellular membrane. Optionally, specific receptors can be included to allow viral particles to bind to the surface preceding fusion. In the case of pH-triggered fusion (e.g., influenza), a dye such as, e.g., a pH-sensitive fluorophore can be incorporated into the bilayer and/or a particle containing one or more viral proteins to monitor the local pH.

In certain exemplary embodiments, one or more lipid bilayers as described herein are immobilized on a support, e.g., one or more high-throughput supports. Suitable supports include, but are not limited to, slides (e.g., microscope slides), beads, chips, particles, strands, gels, sheets, tubing (e.g., microfuge tubes, test tubes, cuvettes), spheres, containers, capillaries, microfibers, pads, slices, films, plates (e.g., multi-well plates), microfluidic supports (e.g., microarray chips, flow channel plates, biochips and the like) and the like. In various embodiments, the solid supports may be biological, nonbiological, organic, inorganic or combinations thereof. When using supports that are substantially planar, the support may be physically separated into regions, for example, with trenches, grooves, wells, or chemical barriers (e.g., lacking a lipid-binding coating). In exemplary embodiments, supports can be made of and/or coated with a variety of materials including, but not limited to glass, quartz, ceramic, plastic, polystyrene, methylstyrene, acrylic polymers, titanium, latex, sepharose, cellulose, nylon, metal (e.g., Au, Pt, Ag, Cu and the like), metal oxide (e.g., AlO₂ and the like) and the like and any combination thereof. Such supports and their uses are well known in the art.

In certain exemplary embodiments, supports may have functional groups on their surface which can be used to attach a lipid bilayer (e.g., a phospholipid bilayer) to the support. For example, at least a portion of the support can be coated with silane and dextran (e.g., high molecular weight dextran). Dextran in its hydrated form can function as a molecular cushion for the membrane and is capable of binding lipids on the support. Suitable functional groups include, but are not limited to, silicon oxides (e.g., SiO₂), MgF₂, CaF₂, mica, polyacrylamide, dextran and the like and any combination thereof.

As used herein, the term “attach” refers to both covalent interactions and noncovalent interactions. A covalent interaction is a chemical linkage between two atoms or radicals formed by the sharing of a pair of electrons (i.e., a single bond), two pairs of electrons (i.e., a double bond) or three pairs of electrons (i.e., a triple bond). Covalent interactions are also known in the art as electron pair interactions or electron pair bonds. Noncovalent interactions include, but are not limited to, van der Waals interactions, hydrogen bonds, weak chemical bonds (i.e., via short-range noncovalent forces), hydrophobic interactions, ionic bonds and the like. A review of noncovalent interactions can be found in Alberts et al., in Molecular Biology of the Cell, 3d edition, Garland Publishing, 1994.

In certain exemplary embodiments, a detectable label is used to label one or more constituents of the compositions and/or assays described herein (e.g., one or more of a viral membrane, an internal portion of a virion, a target membrane and the like). Examples of detectable markers include various radioactive moieties, enzymes, prosthetic groups, fluorescent markers, luminescent markers, bioluminescent markers, metal particles, light scattering nanoparticles, protein-protein binding pairs, protein-antibody binding pairs and the like. Examples of fluorescent proteins include, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride, phycoerythrin, fluorescent CdSe nanocrystals and the like. Examples of bioluminescent markers include, but are not limited to, luciferase (e.g., bacterial, firefly, click beetle and the like), luciferin, aequorin and the like. Examples of enzyme systems having visually detectable signals include, but are not limited to, galactosidases, glucorinidases, phosphatases, peroxidases, cholinesterases, biotins (e.g., biotin-X DHPE) and the like. Identifiable markers also include radioactive compounds such as ¹²⁵I, ³⁵S, ¹⁴C, or ³H. Identifiable markers are commercially available from a variety of sources.

In certain exemplary embodiments, a detectable label is a commercially available fluorophore including, but not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, sulforhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, Cy7 (Amersham Biosciences, Piscataway, N.J.) and the like. FRET tandem fluorophores may also be used, including, but not limited to, PerCP-Cy5.5, PE-Cy5, PE-Cy5.5, PE-Cy7, PE-Texas Red, APC-Cy7, PE-Alexa dyes (610, 647, 680), APC-Alexa dyes and the like. Fluorescent labels are described in many reviews, including Haugland, The Handbook—A Guide to Fluorescent Probes and Labeling Technologies, Tenth Edition (Molecular Probes—Invitrogen Detection Technologies, 2006). In certain exemplary embodiments, novel fluorophores such as Rh110C18 (described further herein) are provided.

In certain exemplary embodiments, fluorescence resonance energy transfer (FRET) is used to monitor fusion (e.g., one or more of hemifusion, pore formation and complete fusion). FRET can be used to measure how close two fluorophores are together. Resonance energy transfer is a mechanism by which energy is transferred directly from one molecule to another, which occurs over a very small distance, usually less than 10 nm. In certain embodiments, a donor particle includes a fluorescent label that acts a donor and a target membrane includes a fluorescent label that acts as an acceptor. In other embodiments, a donor particle includes a fluorescent label that acts an acceptor and a target membrane includes a fluorescent label that acts as a donor. The observance of FRET between donor and acceptor can then be used to quantitate fusion (e.g., one or more of hemifusion, pore formation and complete fusion). FRET methods and compositions are well known in the art and are described in, e.g., Schmid and Sitte (2003) Curr. Opin. Oncol. 15:55.

In certain exemplary embodiments, methods (also referred to herein as a “screening assays”) are provided for identifying modulators, i.e., candidate or test compounds or agents (e.g., peptides, cyclic peptides, peptidomimetics, small molecules, small organic molecules, or other drugs) which alter (e.g., inhibit or stimulate) virus-mediated membrane fusion.

As used herein, the term “small molecule” refers to a molecule, either naturally occurring or synthetic, that has a molecular weight of more than about 25 daltons and less than about 3000 daltons. In certain exemplary embodiments, the small molecule has a molecular weight of less than about 2500 daltons, less than about 2000 daltons, between about 100 to about 1000 daltons, or between about 200 to about 500 daltons.

In one exemplary embodiment, the invention provides assays for screening candidate or test compounds which modulate (e.g., modulators that inhibit or stimulate) virus-mediated membrane fusion. The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412), or on beads (Lam (1991) Nature 354:82), chips (Fodor (1993) Nature 364:555), bacteria (Ladner U.S. Pat. No. 5,223,409), spores (Ladner U.S. Pat. No. 5,223,409), plasmids (Cull et al. (1992) Proc. Natl. Acad. Sci. USA 89:1865) or on phage (Scott and Smith (1990) Science 249:386); (Devlin (1990) Science 249:404); (Cwirla et al. (1990) Proc. Natl. Acad. Sci. USA 87:6378); (Felici (1991) J. Mol. Biol. 222:301); (Ladner supra).

Examples of methods for introducing a molecular library of randomized nucleic acids into a population of cells can be found in the art, for example in U.S. Pat. No. 6,365,344. A molecular library of randomized nucleic acids can provide for the direct selection of candidate or test compounds with desired phenotypic effects. The general method can involve, for instance, expressing a molecular library of randomized nucleic acids in a plurality of cells, each of the nucleic acids comprising a different nucleotide sequence, screening for a cell of exhibiting a changed fusion physiology in response to the presence in the cell of a candidate or test compound, and detecting and isolating the cell and/or candidate or test compound.

In one embodiment, the introduced nucleic acids are randomized and expressed in the cells as a library of isolated randomized expression products, which may be nucleic acids, such as mRNA, antisense RNA, siRNA, ribozyme components, etc., or peptides. The library should provide a sufficiently structurally diverse population of randomized expression products to effect a probabilistically sufficient range of cellular responses to provide one or more cells exhibiting a desired response. In certain exemplary embodiments, at least 10⁶, at least 10⁷, at least 10⁸, or at least 10⁹ different expression products are simultaneously analyzed in the subject methods. In one exemplary embodiment, methods maximize library size and diversity.

The introduced nucleic acids and resultant expression products can be randomized, meaning that each nucleic acid and peptide consists of essentially random nucleotides and amino acids, respectively. The library may be fully random or biased, e.g., in nucleotide/residue frequency generally or per position. In other exemplary embodiments, the nucleotides or residues are randomized within a defined class, e.g. of hydrophobic amino acids, of purines, etc.

Functional and structural isolation of the randomized expression products may be accomplished by providing free (not covalently coupled) expression product, though in some situations, the expression product may be coupled to a functional group or fusion partner, such as, e.g., a heterologous (to the host cell) or synthetic (not native to any cell) functional group or fusion partner. Exemplary groups or partners include, but are not limited to, signal sequences capable of constitutively localizing the expression product to a predetermined subcellular locale such as the Golgi, endoplasmic reticulum, nucleoli, nucleus, nuclear membrane, mitochondria, chloroplast, secretory vesicles, lysosome, and the like; binding sequences capable of binding the expression product to a predetermined protein while retaining bioactivity of the expression product; sequences signaling selective degradation, of itself or co-bound proteins; and secretory and membrane-anchoring signals.

In certain exemplary embodiments, it will be desirable to provide a partner which conformationally restricts the randomized expression product to more specifically define the number of structural conformations available to the cell. For example, such a partner may be a synthetic presentation structure: an artificial polypeptide capable of intracellularly presenting a randomized peptide as a conformation-restricted domain. Generally, such presentation structures comprise a first portion joined to the N-terminal end of the randomized peptide, and a second portion joined to the C-terminal end of the peptide. In certain exemplary embodiments, presentation structures maximize accessibility to the peptide by presenting it on an exterior loop, for example of coiled-coils, (Myszka and Chaiken (1994) Biochemistry 33:2362). To increase the functional isolation of the randomized expression product, the presentation structures are selected or designed to have minimal biologically active as expressed in the target cell. In addition, the presentation structures may be modified, randomized, and/or matured to alter the presentation orientation of the randomized expression product. For example, determinants at the base of the loop may be modified to slightly modify the internal loop peptide tertiary structure, while maintaining the absolute amino acid identity. Other presentation structures include zinc-finger domains, loops on beta-sheet turns and coiled-coil stem structures in which non-critical residues are randomized; loop structures held together by cysteine bridges, cyclic peptides, etc.

In certain exemplary embodiments, an assay is a lipid bilayer-based assay in which a cell which a lipid bilayer is contacted with a test compound and the ability of the test compound to modulate virus-mediated membrane fusion (e.g., inhibit or stimulate one or more steps of virus-mediated membrane fusion) is determined. Determining the ability of the test compound to modulate virus-mediated membrane fusion can be accomplished by monitoring, for example, hemifusion, pore formation and/or complete fusion using one or more assays described further herein.

In exemplary embodiments, novel modulators identified by the above-described screening assays are provided. Accordingly, it is within the scope of this invention to further use a modulator identified as described herein in an appropriate animal model or cell-based assay as described herein. For example, an agent identified as described herein (e.g., a virus-mediate membrane fusion modulating agent) can be used in an animal model or cell-based assay to determine the efficacy, toxicity, or side effects of treatment with such an agent. Alternatively, an agent identified as described herein can be used in an animal model or cell-based assay to determine the mechanism of action of such an agent. In certain exemplary embodiments, uses of novel agents identified by the above-described screening assays for treatments of disorders associated with viral infection (e.g., as a result of virus-mediated membrane fusion) are provided.

In exemplary embodiments, an assay is a cell based assay comprising contacting a cell with a modulator and determining the ability of the modulator to alter virus-mediated membrane fusion (e.g., to alter viral infectivity by inhibiting or stimulating one or more steps of virus-mediated membrane fusion). A cell can be any prokaryotic or eukaryotic cell such as, for example, yeast, bacteria, insect cells, plant cells, reptilian cells, fish cells or amphibian cells (such as Xenopus cells). In certain exemplary embodiments, a cell is a mammalian cell (such as Chinese hamster ovary cells (CHO), mouse cells, African green monkey kidney cells (CV-1, COS), fetal human cells (293T) or other human cells). Other suitable cells are known to those skilled in the art. Both cultured and explanted cells may be used.

In exemplary embodiments, an assay is an animal model based assay comprising contacting an animal with a modulator and determining the ability of the modulator to alter virus-mediated membrane fusion (e.g., to alter viral infectivity by inhibiting or stimulating virus-mediated membrane fusion). In exemplary embodiments, animals include but are not limited to mammals such as non-human primates, rabbits, rats, mice and the like, birds such as chickens and the like, and amphibians such as Xenopus and the like.

The following examples are set forth as being representative of the present invention. These examples are not to be construed as limiting the scope of the invention as these and other equivalent embodiments will be apparent in view of the present disclosure, figures, tables, and accompanying claims. The contents of all references, patents and published patent applications cited throughout this application are hereby incorporated by reference in their entirety for all purposes.

### EXAMPLE I

### Technical Description

Certain aspects of the present invention are based on the methodology described herein that enables one of skill in the art to study the kinetics of fusion of individual viral particles. In this technical description, the method will be illustrated with influenza, the causative agent of the common flu. This method was also successfully tested using vesicular stomatitis virus (VSV), which is the causative agent of vesicular stomatitis in cattle. The fusion of individual, fluorescently labeled particles with a target membrane could be observed using sensitive fluorescence microscopy. Simultaneous use of multiple fluorescent probes enabled observation of different intermediates in the fusion process and characterization of the reaction kinetics. In this section, various novel methods that were developed are described further.

Two different fluorescent reporters could be introduced into the enveloped viral particles, e.g., a lipophilic dye that was embedded in the membrane enveloping the particle and a water soluble dye that is introduced into the core of the viral particle. The synthesis of a novel conjugate of the dye Rhodamine 110 and a lipophilic octadecanol was performed. Fluorescently labeled viral particles were prepared by incubation of the viral particles in a solution containing both dyes. Fluorescent viral particles were then introduced into a microfluidic flow cell containing a supported target bilayer. Upon laser excitation, the fluorescence was imaged onto a CCD camera, allowing the visualization of individual viral particles on the target membrane. The two reporters emitted fluorescence at two different wavelengths, allowing the behavior of the lipids and the content of the individual particles to be followed simultaneously. Tracking of the lipophilic dye allowed the observation of the hemifusion intermediate, and the aqueous content dye reported on completion of the fusion process. Performing these studies at the single-particle level allowed every kinetic step of the fusion process to be observed separately for each particle and resulted in a kinetic characterization of the fusion process with unprecedented accuracy.

### Microscopy

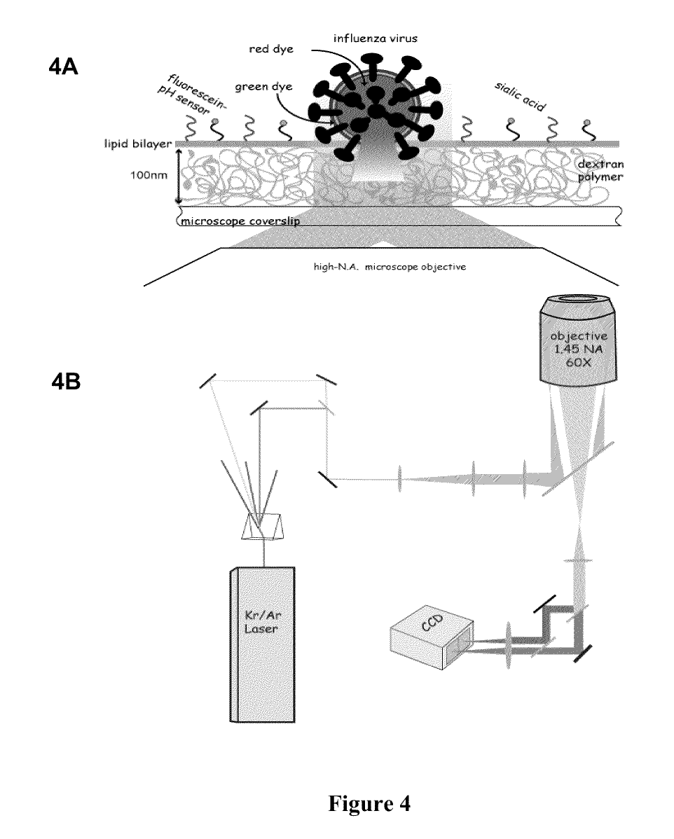

As a target membrane for fusion, a planar lipid bilayer on a glass support was used. The details of bilayer formation are discussed further herein. The thickness of the bilayer and the fact that the events of interest all took place in close vicinity to the membrane rendered it an ideal substrate to be used in total internal reflection fluorescence (TIRF) microscopy. This sensitive mode of imaging allowed for a highly selective laser excitation of only a 100 nm layer immediately above the glass surface (Axelrod (1989) Meth. Cell Biol. 30:245) (FIG. 4A). The efficient suppression of the fluorescence background from bulk solution enabled the visualization of fluorescent particles sufficiently close to the planar target membrane.

All the single particle experiments were performed in home-built, microfluidic flow cells that allowed for easy and fast exchange of reagents (van Oijen et al. (2003) Science 301:1235). The flow cell was placed on an inverted microscope (Nikon TE3000) with an objective of high numerical aperture (N.A.=1.45) that allowed for through-objective-type TIRF microscopy (Ambrose et al. (1999) Cytometry 36:224). An area of approximately 100×100 μm² of the flow cell was illuminated at multiple wavelengths (488 and 568 nm) using an Argon/Krypton laser (Coherent Innova 70C) and the resulting fluorescence was imaged on a sensitive electron-multiplying CCD (Andor Technologies DV 887-BI) at two wavelength regions (510-540 nm and 610-640 nm) (FIG. 4B). The simultaneous excitation and imaging at multiple wavelengths allowed the use of multiple fluorescence probes without the need for switching between filters, resulting in a high time resolution (approximately five msec).

### Bilayer Formation

Two requirements for single-particle observation using fluorescence microscopy are a planar geometry and a fluid support. Conventional liposome-based assays are not easily adapted to single-particle detection, and frictional coupling with glass constrains the bilayer and can interfere with fusion assays. To provide a membrane support that allowed membrane and soluble components to freely diffuse, the glass was functionalized with a thin hydrophilic film (approximately 1 nm thickness) of dextran polymer (Elender et al. (1996) Biosens. Bioelectron. 11:565). In the presence of aqueous buffer, the dextran film hydrated and swelled into a thick, hydrophilic cushion (approximately 100 nm thickness). Since the bilayer could freely move over such a lubricating layer, it exhibited self-healing properties, preventing the formation of defects (Kuhner et al. (1994) Biophys. J. 67:217).

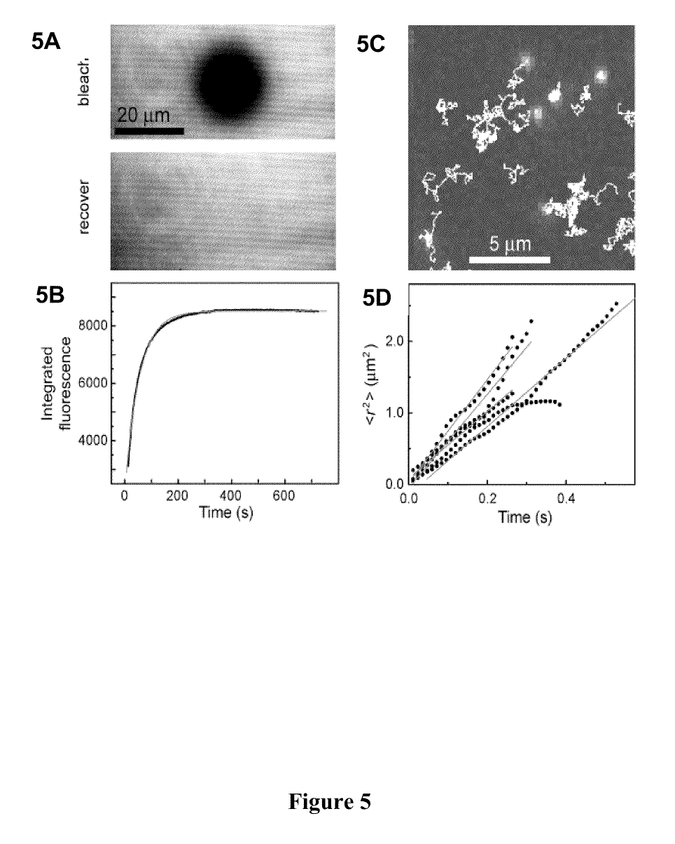

The fluidity of the supported bilayers was tested by introducing a very small amount of fluorescent lipid into the membrane. The two-dimensional diffusion coefficient of the lipids provided a good indication of the quality of the bilayer. The diffusion coefficient was measured in two ways: 1) Fluorescently labeled lipid was introduced into the membrane (1 mol % Texas Red DHPE) and fluorescence recovery after photobleaching (FRAP) was used; and 2) A very low concentration of fluorescent, quantum dot-coupled lipids was introduced into the membrane (10⁻⁷ mol %) and the positions of individual lipids in the membrane were imaged as a function of time (FIG. 5C). A plot of their mean-square displacement versus time provided the two-dimensional diffusion coefficient (FIG. 5D).

Supported lipid bilayers were formed on the dextran cushion from lipid vesicles, which adsorb to the hydrophilic surface and fuse among themselves until they reach critical size and rupture into a planar bilayer (Johnson et al. (2002) Biophys. J. 83:3371; Nollert et al. (1995) Biophys. J. 69:1447). Liposomes containing 80% phosphatidylcholine and 20% cholesterol were made by extrusion through 100 nm pore-size filters. The liposomes were doped with 1% bovine ganglioside GD1a, to provide a receptor for influenza virus. The fluidity of the supported bilayer was assayed by FRAP and by tracking the positions of individual fluorescently labeled lipid molecules over time (FIGS. 5A-5D). In both experiments, the measured diffusion coefficient for the lipid was between 1 and 2 μm²/s, indicating unhindered diffusion that is indistinguishable from fluid bilayers supported on glass (Elender et al. (1996) Biosens. Bioelectron. 11:565; Nollert et al. (1995) Biophys. J. 69:1447; Hovis and Boxer (2001) Langmuir 17:3400). Thus, a fully fluid, planar bilayer with an aqueous reservoir in the dextran, into which the contents of a fusing virion or vesicle can be released has been generated. These experiments demonstrated that the physico-chemical properties of a bilayer supported by a hydrated dextran polymer were much more suitable to study the dynamics of membrane fusion than membranes supported by bare glass. Not only did the membrane retain its fluidity after introduction of the gangliosides required for HA binding, the large water-filled space under the membrane provided more translational freedom to components released from the viral particles.

Both single-lipid tracking and FRAP experiments demonstrated that high-quality, glass-supported bilayers were difficult to assemble in a reliable and reproducible manner. Often, they showed large areas that were less fluid or even immobile. Without intending to be bound by scientific theory, this was likely caused by nonspecific adsorption of the gangliosides to the underlying glass surface. The membrane's close proximity to the glass also complicated detection of viral activity. As discussed further herein, dye was released from the interior of viral particles upon fusion, and detection of fusion activity required that the dye released beneath the membrane was free to diffuse away from the observation area. However, fluorescent dye quickly absorbed to the glass just below the fused virus and effectively cancelled-out any change in fluorescence intensity.

### Internal pH Sensor

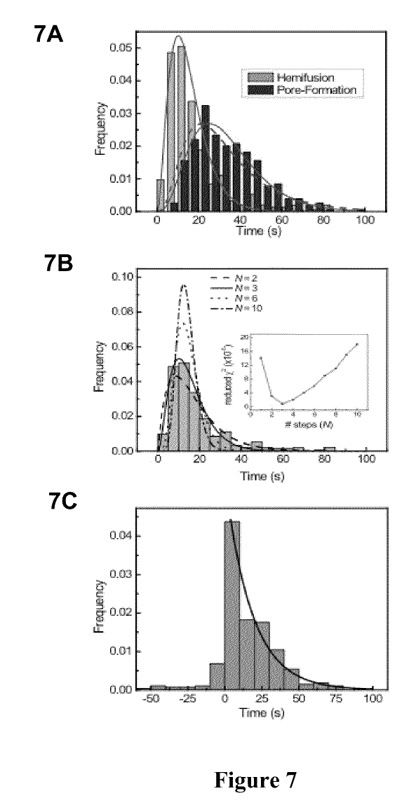

After formation of the membrane in the flow cell and introduction of influenza virus, HA folding and subsequent fusion was initiated by introducing a buffer with low pH (pH 5). To reliably determine at what rates the different fusion events take place, it was necessary to discern exactly when the pH dropped in the vicinity of the membrane. To this end, a low concentration of biotinylated lipids into was introduced into the bilayer (10⁻³ mol %) and the bilayer was incubated with fluorescein-labelled streptavidin. The pKa of fluorescein is 6.4, causing its fluorescence quantum yield to decrease significantly when lowering the pH to a value that induces fusion. The sudden decrease in the green background fluorescence that corresponded to the pH change was clearly visualized in FIGS. 7A and 7B.

### EXAMPLE II

### Detection of Hemifusion at the Single Particle Level

Virus (Influenza A X31, a gift from John Skehel, Mill Hill Laboratories, London, UK) was labelled with the fluorophores Rhodamine-110 octadecyl ester (Rh110C18) and with sulforhodamine B (SRB). The water-soluble SRB label was introduced into the inside of the viral particle (performed as described further herein), and the lipophilic Rh110C18 label was introduced into the viral membrane. The SRB label escaped into the space under the supported bilayer only after fusion was complete and a pore between the viral compartment and the target membrane was formed. The Rh110C18 label contained in the viral membrane escaped into the target membrane when hemifusion occurred and when the two proximal leaflets of the membranes fused into a hemifusion stalk.

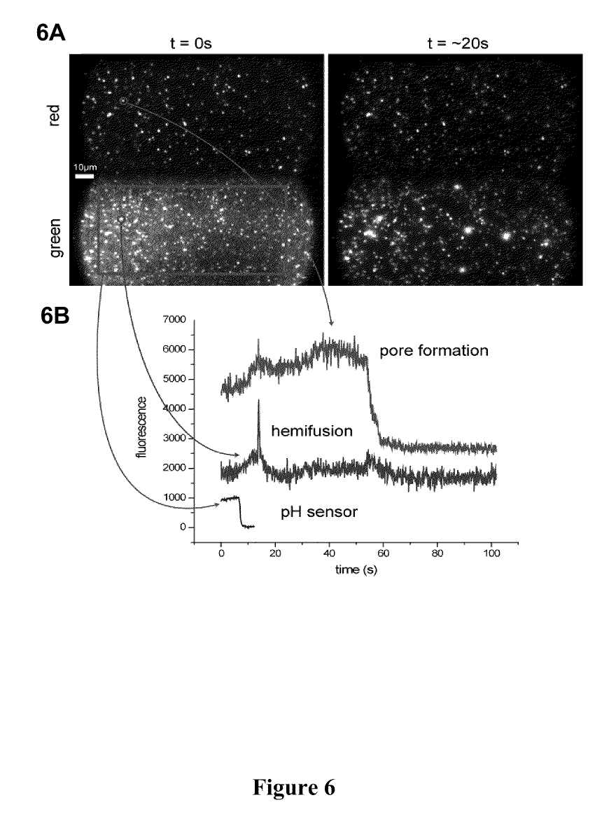

The simultaneous use of two reporters required two distinct emission wavelengths. To this end, use was made of the commercially available, red-emitting SRB label (Aldrich, St. Louis, Mo.) and the synthesized green-emitting R110C18 label. The Rh110C18 label inserted itself from the aqueous phase into membranes and was well suited to label whole viruses without disrupting their structure. The amount of dye added to the virus was equivalent to 6 mol % of the total viral lipid content. This high concentration in the membrane caused the dye to self-quench its fluorescence. Although the quenching significantly reduced the fluorescence from individual viruses, the particles were still clearly visible when anchored to the membrane (FIG. 6A, ‘green’ channel).

A diagram of the experimental setup appears in FIG. 4B. The coverslip bearing the supported bilayer was mounted in a flow cell on the stage of a total-internal reflection fluorescence (TIRF) microscope. Virus particles labeled with Rh110C18 were injected into the flow cell, and attachment to the ganglioside receptors is monitored by appearance of diffraction-limited spots in the image. The lower half of the left panel of FIG. 6A shows an image of the Rh110C18 fluorescence from a 140×70 μm² area of the target membrane. Each bright spot represents a single virus particle bound to the membrane. Omission of ganglioside from the supported bilayer resulted in a 100-fold decrease in the number of docked particles, confirming the specificity of attachment through sialic-acid receptors. Fusion was triggered by lowering the pH in the flow cell from 7.4 to 4.6. Upon lowering the pH, HA inserts its fusion peptide into the target membrane, the HA protein folds, and the viral and target membranes fuse. When hemifusion occurred, the Rh110C18 label could freely diffuse from the viral membrane into the target membrane, causing a rapid decrease of the local dye concentration and resulting in a dequenching of the fluorescence. This rapid increase of signal was followed by a gradual decrease caused by the radial diffusion of the dye into the planar target membrane. At the single-particle level, hemifusion was clearly visible as an instantaneous increase in brightness of a membrane-anchored viral particle, followed by a cloud-like expansion of the dye (‘green’ channel in FIG. 6A; ‘hemifusion’ trace in FIG. 6B). Upon completion of the fusion process, the red SRB label escaped the particle and diffused outwards, corresponding with a decrease of the red fluorescence (FIG. 6B, ‘pore formation’ trace).

For precise determination of the time at which the pH drop reached any particular virion, a low concentration of biotinylated lipid was introduced into the bilayer (10⁻³ mol %), to capture fluoresceinated streptavidin. The low background of fluorescein thus created served as a detector for the shift in pH, as fluorescein adopts a non-fluorescent configuration below pH approximately 6.4 (Klonis et al. (1998) Photochem. Photobiol. 67:500). The sudden decrease in the green background fluorescence that corresponded to the pH change was clearly visualized in FIGS. 6A and 6B. The time to hemifusion of any single particle can thus be determined by measuring the elapsed time between the drop in fluorescein background and the de-quenching of Rh110C18.

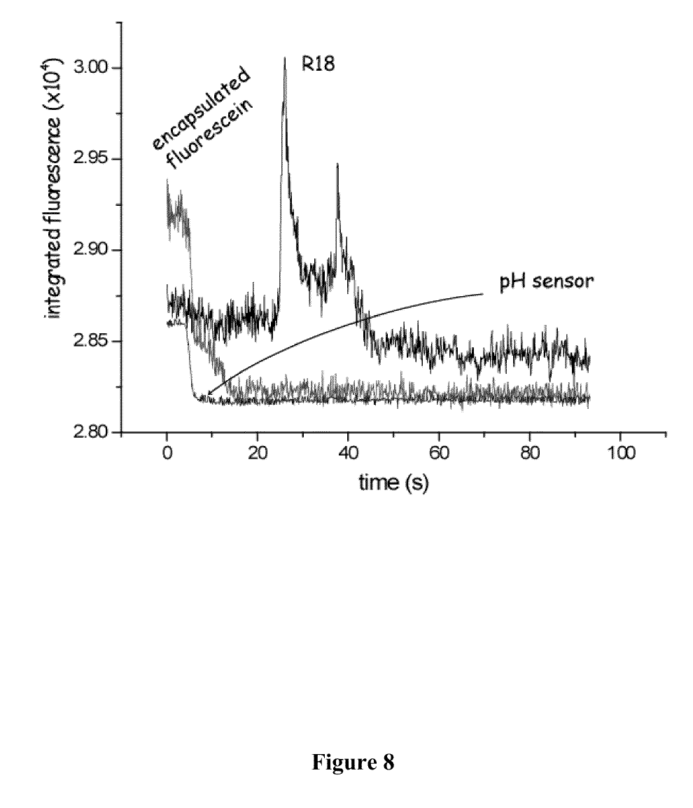

FIG. 7A depicts a histogram of the times elapsed between the pH drop, hemifusion and fusion for each of 2,138 individual viral particles. This histogram provided a very direct view on the various rates involved in hemifusion formation. The rise and decay in the histogram indicated that an intermediate was formed before lipid mixing occurred. The data could be fitted using the algorithms described further herein. FIG. 7B shows the time lapsed between hemifusion and full fusion for each of 296 individual particles. Importantly, this distribution, reporting on the lifetime of the hemifusion intermediate, is impossible to obtain using conventional, ensemble-averaged techniques and is important in characterizing the effect of drugs that inhibit progression of the fusion process to pore formation. FIG. 7C shows the distribution of lag times between hemifusion and pore formation of individual viral particles. FIG. 8 depicts the detection of proton influx prior to hemifusion.

### pH Dependence of Hemifusion

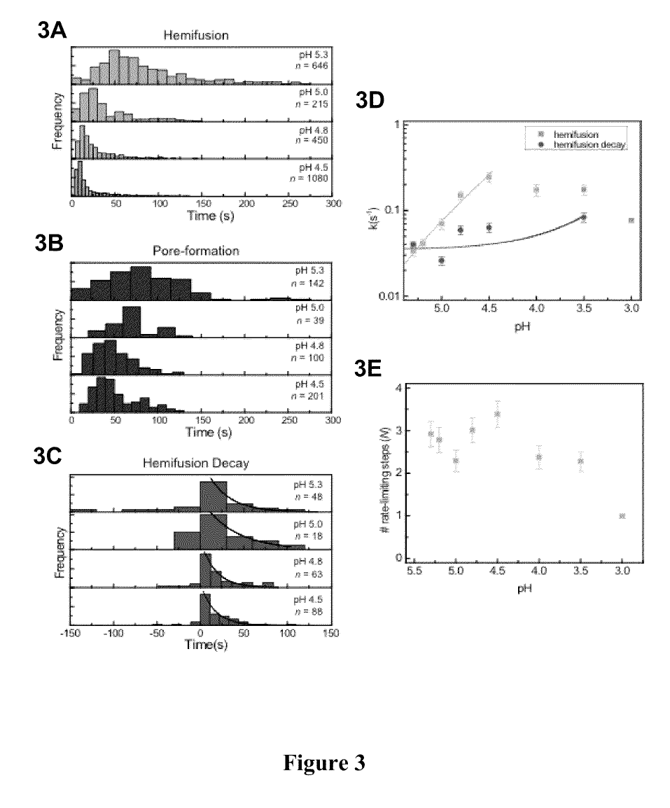

To explore the mechanism of pH activation, the pH of the activating buffer was varied. As expected, the lag time between the pH drop and hemifusion increased with increasing pH (FIGS. 3A and 3B). The hemifusion lifetime was relatively independent of pH, however, and the rate constant for the fusion step changed by less than threefold between pH 4.5 and 5.3 (FIGS. 3C and 3D). The pH insensitivity indicated that virus particles are already committed to fusion once they have reached the hemifusion intermediate.

Gamma-distribution analysis of hemifusion kinetics in the pH range between 4.5 and 5.3 showed that the number of steps remains constant at about N=3, and that the individual rate constants varied in parallel (FIGS. 3D and 3E). If proton binding were part of each of these (apparently identical) three steps, then they should continue to depend smoothly on proton concentration as the pH drops. But instead, the rate constant leveled off, indicating that the extent of proton binding might determine the effective concentration of a species, which then undergoes the actual, rate-limiting rearrangement. Below pH 4.7 or so, the concentration of proton-bound HA species was no longer an issue, and in that regime the actual rearrangement rate might determine the apparent kinetic constant. Both k and N dropped sharply below pH 3.5, probably reflecting low-pH induced inactivation or denaturation of HA.

### EXAMPLE III

### Fusion Pore Formation

To label the interior of the influenza virions, the virus preparation was incubated overnight in a concentrated (10 mM) solution of SRB. Excess dye was then removed by gel filtration. SRB penetrated the virion bilayer and accumulated in the viral interior. Dye loaded virus was used promptly, to avoid loss by back diffusion. The interior labeling procedure was combined with Rh110C18 membrane labeling to produce doubly labeled particles. Co-localization analysis showed that among the membrane-docked, fluorescent particles, 90% contained Rh110C18, 40% contained SRB, and 30% contained both dyes.

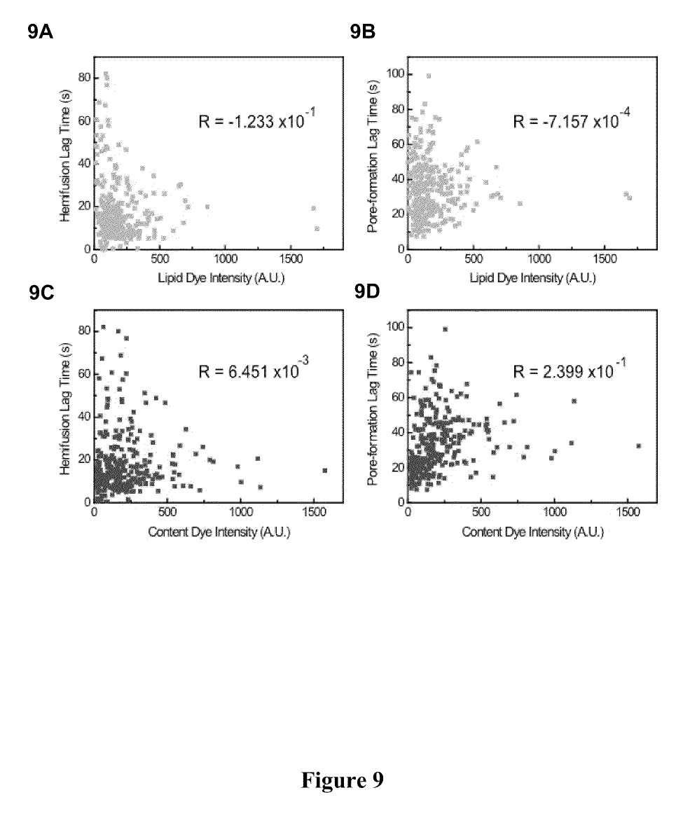

The upper half of FIG. 6A shows the fluorescence of the SRB from the same 140×70 μm² area of the target membrane as depicted in the lower half. The red trace in FIG. 6A shows rapid decay of the SRB signal of the same particle for which the hemifusion trace is shown. Loss of red content signal starts several seconds after the R110C18 de-quenching burst. The decay reported loss of SRB from the virion following fusion pore formation, as the dye diffused into the fluid support of the bilayer. Thus, the time elapsed between hemifusion and fusion pore formation could readily be obtained for each particle. Hemifusion kinetics were not affected by addition of the interior dye, nor were kinetics of fusion pore formation altered by the presence of SRB in the viral membrane (FIG. 9).

### EXAMPLE IV

### Intermediate States

The times elapsed between pH drop and hemifusion and between pH drop and fusion were determined by locating the maximum and minimum slopes in the single-particle traces for Rh110C18 and SRB (FIG. 7B). FIG. 3A depicts the distribution of lag times for hemifusion and pore formation (n=309) compiled from experiments conducted at 23° C. and pH 4.6. Both histograms show a rise and decay in the frequency of events, indicating intermediate states. Thus, without intending to be bound by scientific theory, even the first event (hemifusion) could not have been a single-step transition, or an exponentially distributed lag time would have been observed. A simple kinetic model describes a multi-step transition as series of N intermediates between initial and final states, with a single rate constant, k₁, for each transition:

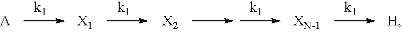

where A is the initial configuration at t=0, the time of the pH drop, and H is the hemifused state at time t. The probability density for this scheme is a gamma distribution:

$${p}_{H} ( t ) = \frac{{k}_{1}^{N} {t}^{N - 1}}{Γ ( N )} {e}^{- {k}_{1} t} .$$

Use of this expression to fit the hemifusion lag times (FIG. 7A) yields k₁=0.20±0.02 sec⁻¹ and N=3.1±0.2. Fits were also shown with k₁ as the only free parameter and N fixed at 2, 3, 6, or 10 (FIG. 7B). The inset in FIG. 7B shows the χ² goodness-of-fit as a function of N, with a clear minimum at N=3.

The pore-formation lag-time distribution could be fit by a gamma distribution with N=4 (solid red curve in FIG. 7A), indicating a single step from hemifusion to fusion. A more direct analysis took advantage of the determination of hemifusion and fusion from the same particle, thus allowing one to determine the distribution of time intervals between the two events. Among the particles that contained both lipid and content dyes, about 10% showed both hemifusion and fusion signals. For these particles, the distribution of hemifusion lifetimes (the time between hemifusion and pore formation) is shown in FIG. 7C. In 90% of the traces, de-quenching of Rh110C18 (hemifusion) preceded loss of SRB signal (fusion), consistent with the assumption that a hemifused membrane was an essential intermediate, rather than an abortive, off-pathway state (Zimmerberg et al. (1994) J. Cell. Biol. 127:1885; Chernomordik et al. (1998) J. Cell. Biol. 140:1369). A single exponential decay (k₂=0.55±0.004 sec⁻¹) fit the positive lag times, consistent with the conclusion that the transition from hemifusion to pore-formation involved a single, rate-limiting step. Furthermore, the pore formation lag-time distribution could be described by a convolution of the N=3 gamma distribution of hemifusion times with the experimentally observed single-exponential transition between hemifusion and pore formation (FIG. 7A, dashed red curve). These results were all consistent with three intermediate states before hemifusion and a single rate-limiting step between hemifusion and pore formation.

### EXAMPLE V

### Discussion

Observation of individual influenza A particles fusing with a target membrane has allowed detailed information on the kinetics of both hemifusion and pore formation to be obtained. Analysis of hemifusion revealed about three intermediate steps preceding formation of a hemifusion stalk. In previous studies of HA-expressing cells fusing with red blood cells, Danielli et al. found a time lag between acidification and initial detection of fluorescent dye redistribution and interpreted this result in terms of the accumulation of hidden intermediates (Danieli et al. (1996) J. Cell Biol. 133:559). Subsequent analyses have usually relied on a similar experimental format (Leikina et al. (2002) EMBO J. 21:5701; Mittal et al. (2002) Biophys. J. 83:2652; Mittal, A., Leikina, E., Chernomordik, L. V. & Bentz, J. (2003) Biophys. J. 85, 1713-1724). The large number of individual fusion events that make up the observed signal in such an experiment quickly lose coherence, essentially blurring the kinetic information. The single-particle studies made possible by the experimental design we describe give a more direct view of the kinetics, without any dephasing, and thus permit estimates of the number of intermediates and of the rates of the transitions between them.

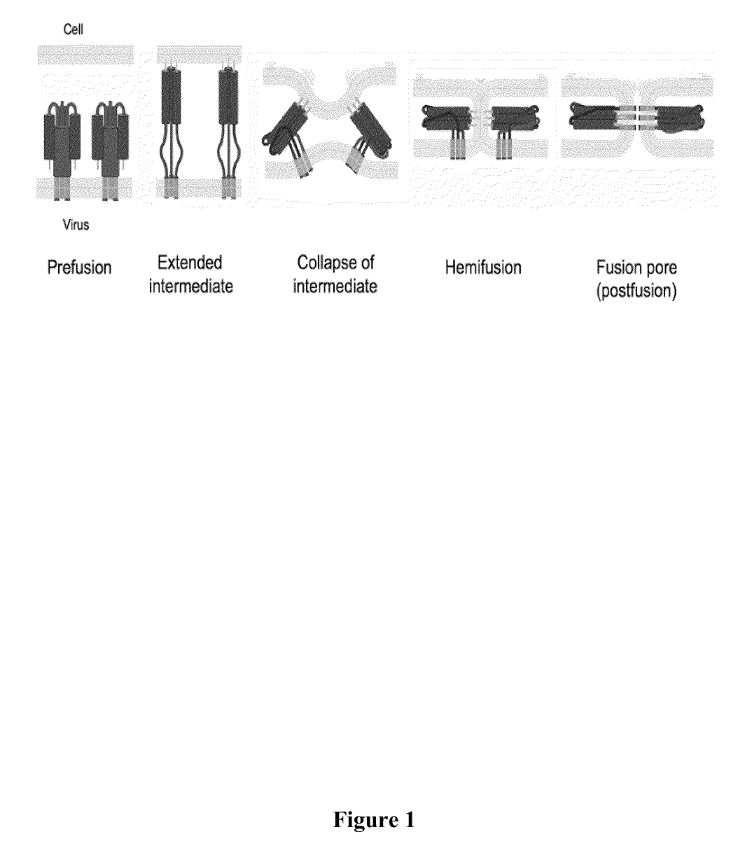

Direct evidence for a molecular description of the multiple rate-limiting intermediates preceding hemifusion has not yet been obtained. The current picture of fusion as mediated by HA and other class-I viral fusogens involves formation of an extended or “prehairpin” intermediate—an extended trimer with its fusion peptides inserted into the target-cell membrane and its TM segments anchored in the viral bilayer (FIG. 1, second panel). The conformation of HIV-1 gp41 that is sensitive to peptide inhibitors such as T-20 is generally agreed to have such an extended structure; comparable data are not yet available for HA. Without intending to be bound by scientific theory, it would be expected that formation of an HA extended intermediate would depend on pH, while the subsequent steps—fusion-peptide insertion and collapse (foldback) to the postfusion structure—might well be pH-independent. Thus, in the higher pH range we have examined (i.e., above approximately pH 4.5), the pH-dependence of hemifusion indicates that the rate-limiting step is the formation (or clustering) of enough activated timers within the area of contact of virus and target bilayer to promote approach of the two membranes, while below approximately pH 4.5, collapse of clustered (load-bearing) HA₂ near the fusion site probably becomes the slowest process. The pH at which activation is no longer limiting agrees well with the pH at which the measured rate of HA conformational change becomes very rapid, as measured by fluorescence changes in detergent-solubilized HA from the same, X31, influenza strain (Krumbiegel et al. (1994) Biophys. J. 67:2355).

The model underlying the gamma distribution fit used here to analyze hemifusion kinetics assumes N sequential steps, each with the same rate constant. The value of N is thus a formal rather than literal parameter, as various simplifying conditions have been imposed. A relatively uniform value for N (about 3) for pH>4.5 was obtained. At pH<4.5, the process (suggested to be exposure and activation of HA₂) that is rate-limiting at higher pH will still make a substantial contribution (see extrapolation in FIG. 5D), and only at very low pH (<3), where inactivation by denaturation may also be occurring, would it have been possible to interpret N and k₁ purely in terms of post-activation steps. Thus, the value of N at pH<4.5 is inevitably a complex average of two different stages in the reaction. In the higher pH range, we can probably take N as a lower bound on the number of HA trimers required for fusion. A three-HA minimal fusion model is consistent with the conclusions of Danielli et al., who found that the lag time between pH drop and redistribution of R18 from HA-expressing cells had a power-law dependence (with N=3) on the average number of HA trimers per cell (Danieli et al. (1996) J. Cell Biol. 133:559). Others have attempted to differentiate between the number of HA molecules in a fusion aggregate and the number actually activated by proton binding, one estimate being about 8 for the former number and 2 for the latter (Mittal et al. (2002) Biophys. J. 83:2652).

Whatever the precise meaning of N˜3, a model with several sequential steps clearly fits the observed hemifusion kinetics far better than any single-step model. If the pH-dependent step that dominates the kinetics above approximately pH 4.5 includes the various reorganizations involved in extracting the fusion peptide from its pocket and spreading apart the HA₁ globular domains, then the sequential model indicates that completion of one such rearrangement is a requirement for the next. If at the start of the process, essentially all HA₁ subunits at the interface between virus and membrane are bound to a ganglioside, a transition to the extended intermediate in one trimer might “push” the target membrane away from the virion, permitting extension of a neighboring trimer. This sort of phenomenon has been proposed to explain values obtained in the kinetic analysis offered by Mittal et al. Id. The relevant parameter in their model, k_{f}, is about 0.1-0.2 sec⁻¹, of just the same magnitude as our parameter, k₁.

If one assumes that at pH 3.5-4, the rate of hemifusion reflects primarily the rate of extended intermediate collapse, then the lifetime of this intermediate is about 15-20 seconds. The prehairpin intermediate of gp41 has a much longer lifetime (many minutes), as indicated by the “window of opportunity” for T-20 inhibition following association of gp120 and CD4 (Chan (1998) Cell 89:681). The balance between the membrane tension against which collapse of the extended intermediate must act and the free energy gained by zipping up of the outer layer structure against the inner-layer coiled-coil may determine these lifetimes.

The types of observations made so far limit the detail with which we can trace the fusion pathway, but the advantages of observing fusion of individual virions with a defined and homogeneous membrane are evident. Without intending to be bound by scientific theory, the defined contact area likely includes a uniform patch of 20-30 HA trimers, depending on the deformability of the fluid supported bilayer. Gangliosides can serve as authentic influenza virus receptors (Herrler and Klenk (1987) Virology 159:102). The ganglioside density in the supported bilayers described herein was about one per 60 nm², within the range of concentration for glycosphingolipids in various cell types. Gangliosides will probably tend to cluster through local phase separations (Ferraretto et al. (1997) Biochemistry 36:9232; Menke et al. (2002) Eur. Biophys. J. 31:317), and rapid lateral diffusion is in any case likely to ensure that most HA₁ subunits within the contact region are attached to the membrane. Thus, the way the virus “sees” the bilayer in these experiments likely resembles the situation within an acidifying endosome more closely than in previously available assays. We therefore believe that we have developed a useful format in which to dissect the mechanism of viral membrane fusion.

As the first step in viral infection, viral fusion is a valuable drug target. Blocking viral membrane fusion will prevent the viral particle from entering and infecting cells before the viral genome is copied and packaged into large numbers of progeny virus.

### EXAMPLE VI

### Experimental Protocols

### Surface Functionalization

### Cleaning

Glass microscope cover slips (25×25 mm No. 1.5, VWR, West Chester, Pa.) were cleaned in a bath sonicator while immersed successively in detergent, one molar potassium hydroxide, ethanol, and acetone. Sonication was carried out for thirty minutes in each solvent, and the cover slips were rinsed in 18.2 MΩ deionized water after each sonication step. The cover slips were then submerged in a 3:1 solution of sulfuric acid and 30% hydrogen peroxide for 15 minutes. This final cleaning step served to remove any remaining organic residue from the glass and leave a uniform hydrophilic surface. After a final water rinse, the cover slips were dried in a laboratory oven at 120° C.

### Silane and Dextran Deposition

The cover slips were agitated for five minutes in a 0.2% (v/v) solution of (3-glycidooxypropyl)trimethoxysilane in isopropanol, followed by rinsing in additional isopropanol to remove excess silane. The adsorbed silane layer was cured for one hour at 80° C. Dextran 500 (G.E. Healthcare, Waukesha, Wis., mean molecular weight: 5×10⁵) was dissolved in deionized water to make a 30% solution (3 g/10 ml), and the silanized cover slips, arranged on a flat surface, were covered with approximately one milliliter of this solution. The reaction was left undisturbed for 24-36 hours in a sealed container to prevent evaporation. Unreacted dextran was removed by soaking the cover slips for 48 hours in deionized water. Finally, the dextran functionalized cover slips were dried and stored in a vacuum dessicator.

### Microfluidic Flow Cell Fabrication

Flow cell channels were constructed by cutting a 15×2 mm channel into a 20×20 mm piece of double-stick tape (Grace Bio-Labs, Bend, Oreg.). The tape was then sandwiched between a dextran functionalized cover slip and a 20×20×1 mm fused quartz microscope slide with holes drilled at either end of the channel. Twenty centimeter lengths of polyethylene tubing (INTRAMEDIC™, BD, Franklin Lakes, N.J., I.D.=0.38 mm) were inserted into these holes, and the flow cell was sealed with epoxy glue. For experiments conducted at elevated temperatures, the flow cells were modified by replacing the quartz slide with a thicker 20×20×3 mm piece that was machined to incorporate a thermocouple at the top surface of the channel.

### Supported Bilayer Preparation

Liposomes were composed of a 4:4:2:0.1:5×10⁻⁵ ratio of 1,2,dioleoyl-sn-glycero-3-phosphocholine (DOPC, Avanti Lipids, Alabaster, Ala.), 1-oleoyl-2-palmitoyl-sn-glycero-3-phosphocholine (POPC, Avanti Lipids), cholesterol (Avanti Lipids), bovine brain disialoganglioside GD₁ₐ (Sigma, St. Louis, Mo.), and N-((6-(biotinoyl)amino)hexanoyl)-1,2-dihexadecanoyl-sn-glycero-3-phosphoethanolamine (biotin-X DHPE, Molecular Probes, Eugene, Oreg.). Lipids were mixed in a test tube as chloroform/methanol solutions, and the solvent was removed by evaporation under a stream of nitrogen gas. Residual solvent was removed from the lipid film by placing the test tube in a vacuum desiccator for at least two hours. The dried lipid film was then suspended by gentle agitation in HNE buffer (5 mM HEPES, 145 mM NaCl, 0.1 mM EDTA and 0.01% NaN₃) at a concentration of approximately 10 g/L, and liposomes were extruded through a polycarbonate membrane filter with a pore size of 100 nm.

Planar supported bilayers were formed over hydrated dextran surfaces by the vesicle-spreading method (Nollert et al. (1995) Biophys. J. 69:1447). Liposomes were drawn into the flow cell and incubated for thirty minutes. During this time, liposomes adsorbed to the surface fused with neighboring liposomes until they ruptured and spread over the surface to form a lipid bilayer.

### Rhodamine 110 Octadecyl-Ester (Rh110C18) Synthesis

Specifically, preparation of rhodamine 110 octadecyl ester trifluoroacetate salt was performed as follows. A mixture of 30 mg (0.08 mmol) of rhodamine 110 chloride salt and 7.5 g octadecanol under Ar was heated in an oil bath which had been pre-heated to 80° C. until complete melting of the octadecanol had been observed. 0.1 mL concentrated sulfuric acid was then added and the reaction mixture was allowed to stir for 48 hours after which 0.6 mL dry triethylamine was added dropwise over the course of 5 minutes. Upon completion of triethylamine addition, the reaction mixture was allowed to cool to 25° C. The resulting solid was suspended in 100 mL diethyl ether and stirred vigorously for 40 minutes. Filtration of the resulting suspension afforded approximately 1.5 g of a bright red solid that was subjected to column chromatography. A slurry of 2 g silica gel in 10% isopropanol in chloroform was added to a column with a diameter of 3 cm. The silica plug was then topped off with a slurry of 20 g neutral alumina in 10% isopropanol in chloroform. The compound was eluted with 100 mL each of 10%, 20%, 30% and 40% isopropanol in chloroform followed by 300 mL of 10% methanol plus 20% isopropanol in chloroform. Evaporation of UV-active fractions afforded 33 mg of a bright red solid which was further subjected to HPLC (10 mL/min. on a C4 prep. column; 10 min. at 10% isopropanol and 0.1% TFA in water followed by a 45 min. ramp to 90% isopropanol and 0.1% TFA in water). The resulting solid was washed with 20 mL ether to afford 28 mg (50%) of a red solid. ¹H NMR (600 MHz, CD3OD): 0.90 (t, 3H, J=7.2 Hz); 0.94 (m, 2H); 1.13 (m, 4H); 1.22-1.33 (m, 28H); 3.94 (t, 2H, J=6.3 Hz); 6.81-6.84 (m, 4H); 7.06 (d, 2H, J=9.0 Hz); 7.42 (d, 1H, J=7.2 Hz); 7.81 (dt, 1H, J=7.8 Hz, J=1.8 Hz); 7.85 (dt, 1H, J=7.8 Hz, J=1.8 Hz); 8.30 (d, 1H, J=7.8 Hz). ¹³C NMR (150 MHz, CD3OD): 165.7; 160.2; 160.1; 158.5; 133.4; 132.7; 131.7; 131.1; 130.7; 130.3; 116.8; 113.8; 97.3; 65.6; 31.9; 29.6; 29.54; 29.51; 29.48; 29.30; 29.26; 29.1; 28.2; 25.8; 22.2; 13.2. The rhodamine ester-trifluoracetate salt is shown below.

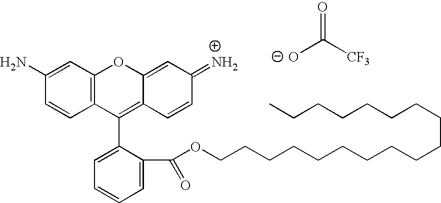

### Labeling and Purification of Viral Particles

Influenza particles were labeled with sulforhodamine B (SRB; Aldrich) and rhodamine 110 octadecyl ester (Rh110C18). Ten microliters of influenza virus (approximately 10 μg viral protein) was mixed with 20 μL of 20 mM SRB in HNE buffer, and left at room temperature for 16-20 hours. Unincorporated dye was separated from the virus with a gel filtration column (PD-10 desalting column, G.E. Healthcare) in a total volume of 0.8 ml. A 2 mM solution of Rh110C18 was prepared in dimethylformamide, and 13 μL was added to the SRB labeled virus particles and agitated for three hours. The virus was eluted from a second PD-10 column before use in the fusion assay.

### Microscope Configuration

Single particle fusion assays were conducted on an inverted fluorescence microscope (Nikon TE-2000U) with a high numerical aperture objective (N.A.=1.45). Viral particles were illuminated with an Argon/Krypton laser (Innova 70C, Coherent, Santa Clara, Calif.) operating in the multi-line mode. The ‘white’ beam emitted from the laser was dispersed with a series of two equilateral prisms. The separated 488 nm and 568 nm lines were band-pass filtered (Chroma Technology Corp., Rockingham, Vt.) and combined with a dichroic mirror before being focused on the back focal plane of the objective. This focusing lens was mounted on a translation stage in order to align the beam along the outer edge of the objective's back aperture and thereby achieve the critical incident angle for total internal reflection at the glass/water interface. Fluorescence emission was collected with the objective and filtered through a 500-540 nm and 600-640 nm dual band-pass emission filter (Chroma Technology Corp.). Outside the microscope, the emission light was collimated and split into separate green and red channels using another dichroic mirror, and each channel was focused separate regions of an electron multiplying CCD camera (DV 887-BI, Andor Technologies, Belfast, Great Britain).

### Execution of the Fusion Assay

A flow cell with a prepared supported membrane was mounted to the microscope stage and coupled to a peristaltic pump (VWR) calibrated to draw buffer at a rate of approximately 0.1 ml/min. Labeled virus diluted to approximately 50 ng/ml and pumped into the flow cell until the surface was saturated or the desired particle density had been reached. Fluorescein-labeled streptavidin (Molecular Probes) was diluted to 2 μg/ml in HNE buffer and then pumped into the flow cell for two minutes followed by a one minute wash with HNE buffer. The fusion reaction was then initiated by flowing an acidic buffer containing 10 mM citric acid, 140 mM NaCl, 0.1 mM EDTA and 0.01% NaN₃. Time-lapsed fluorescence images were recorded at 10 Hz for 200-400 seconds using Andor IQ imaging software.

For experiments conducted at elevated temperatures, applying heat to the flow cell was not sufficient as the oil immersion objective acts as a very efficient heat-sink. Therefore, a resistive band-heater and digital temperature controller were used to heat the microscope objective (Bioptechs, Inc., Butler, Pa.). A thermocouple in contact with the objective provided temperature feedback to the controller, and an additional thermocouple incorporated into the flow cell allowed adjustment of the objective heater to reach the desired temperature within the flow cell.

### Data Analysis

All data was analyzed with software developed in-house and written in Matlab.

### Particle Detection and Fluorescence Intensity Extraction

Ten images acquired prior to onset of fusion were averaged and band-pass filtered to remove noise and background intensity. Particles were located by calculating the center-of-mass positions of fluorescent regions. In order to identify red and green fluorescence intensities corresponding to individual viral particles, the image was divided into red and green channels and the 2-D cross-correlation was calculated from the position coordinates in each channel. The offset corresponding to the maximum correlation was then used to match the red and green intensities of each particle. Fluorescence trajectories were calculated by integrating the intensities from a 4×4 pixel region around each particle.

### pH Sensor

The background intensity of the green channel was integrated to measure the emission from the fluorescein-streptavidin pH sensor. When acidic buffer reached the membrane surface, the fluorescence quantum yield of the surface bound fluorescein significantly decreased and resulted in a drop in fluorescence. The time of the pH drop was estimated from the integrated fluorescence trajectory by finding the intersection of a best-fit line through the base-line and the tangent-line passing through the point with the minimum slope.

### Fusion Event Detection

Trajectories were plotted and manually selected for particles showing fusion activity. Hemifusion and pore formation were defined as the maximum rates of fluorescence increase or decay. In order to facilitate event detection, each trajectory was transformed using a first-order Savitzky-Golay differentiation filter. The maximum or minimum values of the resulting time-derivative traces yielded the respective hemifusion and pore formation times.

Event times were recorded and plotted as hemifusion and pore formation probability distribution histograms. Additionally, the lag time between hemifusion and pore-formation was calculated for each viral particle and plotted.

### Kinetic Model and Non-Linear Least Squares Fitting

The kinetic model of fusion experiments described herein assumed the progression of the virus through a series of irreversible intermediate steps followed by single steps to hemifusion and pore formation:

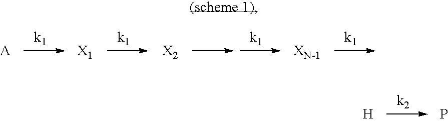

where A is the pre-fusion state, X represents the intermediate kinetic states, and H and P represent the hemi-fused and fused states, respectively. Lowercase letters indicate the kinetic rate constants for each state.

Solving the corresponding differential equations for an indeterminate number of intermediate steps yielded the following expressions:

$$\begin{matrix}{p}_{H} ( t ) = \frac{{k}_{1}^{N} {t}^{N - 1}}{Γ ( N )} {e}^{- {k}_{1} t} & ( 1 ) \\ {p}_{Pore} ( t ) = \frac{{k}_{2} {k}_{1}^{N}}{{( {k}_{1} - {k}_{2} )}^{N}} \frac{Γ ( N , ( {k}_{1} - {k}_{2} ) t )}{Γ ( N )} {e}^{- {k}_{2} t} & ( 2 ) \\ {p}_{H \to P} = {k}_{2} {e}^{- {k}_{2} t} . & ( 3 )\end{matrix}$$

Equations (1) and (2) describe the probability distribution functions for hemifusion and pore formation. N is the number of intermediate steps, and Γ(N) and Γ(N, . . . ) are the gamma and lower-incomplete gamma functions. Equation (3) describes the probability of decay from the hemifusion state to full fusion.

The fusion rate constants were estimated by minimizing the error of N, k, a, and b (Eq. 2 and 3 only) against the experimental time distributions using a non-linear least squares fitting algorithm in Matlab or OriginLab.

### Theory

Multi-exponential fusion kinetics arise from a reaction scheme in which the virus passes through N sequential steps prior to hemifusion:

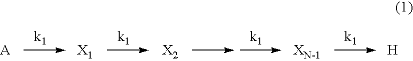

Each transition to the next intermediate step is a random (Poisson) process, and the probability of turnover at time t is k·exp(−kt). The overall hemifusion probability density function consists of the convolution of each intermediate step. Since convolution is equivalent to multiplication in the frequency domain, the Laplace transform was preferred for use:

$$\tilde{P} ( s ) = {\int }_{0}^{∞} p ( t ) {e}^{- st} d t = \frac{1}{s + {k}_{1}} , \qquad ( 2 )$$

where s was the Laplace variable. The transformed hemifusion probability density function was then the product of each step:

$${\tilde{P}}_{H} ( s ) = {( \frac{1}{s + {k}_{1}} )}^{N} . \qquad ( 3 )$$

Finally, the hemifusion probability density function was obtained by transforming back to the time domain:

$${p}_{H} ( t ) = \frac{1}{2 π i} {\int }_{0}^{i ∞} {\tilde{P}}_{H} ( s ) {e}^{st} d s = \frac{{k}_{1}^{N} {t}^{N - 1}}{Γ ( N )} {e}^{- {k}_{1} t} . \qquad ( 4 )$$

The transition from the hemifusion intermediate to pore formation was modeled as a single exponential decay:

p_{H→P}(t)=k₂e^{-k}^{₂}^{t} (5).

The overall lag-time from pH drop to pore-formation was a convolution of the hemifusion lag time and the hemifusion lifetime distributions (or a linear combination in the Laplace domain):

$$\begin{matrix}\begin{matrix}{p}_{P} ( t ) = \frac{1}{2 π i} {\int }_{0}^{i ∞} {\tilde{P}}_{H} ( s ) {\tilde{P}}_{H \to P} ( s ) {e}^{st} d s \\ = \frac{{k}_{2} {k}_{1}^{N}}{{( {k}_{1} - {k}_{2} )}^{N}} \frac{Γ ( N , ( {k}_{1} - {k}_{2} ) t )}{Γ ( N )} {e}^{- {k}_{2} t} ,\end{matrix} \\ where & ( 6 ) \\ {\tilde{P}}_{H \to P} ( s ) = \frac{1}{s + {k}_{2}} . & ( 7 ) \\ Γ ( N ) = {\int }_{0}^{∞} {t}^{N - 1} {e}^{t} d t , \\ and & ( 8 ) \\ Γ ( N , ( k 1 - k 2 ) t ) = {\int }_{0}^{( k - a ) t} {t}^{N - 1} {e}^{t} d t & ( 9 )\end{matrix}$$

were the gamma and lower incomplete gamma functions, respectively.

### EXAMPLE VII

### References

1. Chernomordik, L. V. & Kozlov, M. M. (2003) Annu. Rev. Biochem., 72:175-207.
2. Harrison, S. C. (2005) Adv. Virus Res., 64:231-261.
3. Skehel, J. J. & Wiley, D. C. (2000) Annu. Rev. Biochem., 69:531-569.
4. Matlin, K. S., Reggio, H., Helenius, A. & Simons, K. (1981) J. Cell. Biol., 91:601-613.
5. Wilson, I. A., Skehel, J. J. & Wiley, D. C. (1981) Nature, 289:366-373.
6. Bullough, P. A., Hughson, F. M., Skehel, J. J. & Wiley, D. C. (1994) Nature, 371:37-43.
7. Furuta, R. A., Wild, C. T., Weng, Y. & Weiss, C. D. (1998) Nat. Struct. Biol., 5:276-279.
8. Rimsky, L. T., Shugars, D. C. & Matthews, T. J. (1998) J. Virol., 72:986-993.
9. Frey, G., Peng, H., Rits-Volloch, S., Morelli, M., Cheng, Y. & Chen, B. (2008) Proc. Natl. Acad. Sci. USA, 105:3739-3744.
10. LeNeveu, D. M., Rand, R. P., Gingell, D. & Parsegian, V. A. (1976) Science, 191:399-400.
11. Chanturiya, A., Chernomordik, L. V. & Zimmerberg, J. (1997) Proc. Natl. Acad. Sci. USA, 94:14423-14428.
12. Chernomordik, L. V., Leikina, E., Frolov, V., Bronk, P. & Zimmerberg, J. (1997) J. Cell. Biol., 136:81-93.
13. Danieli, T., Pelletier, S. L., Henis, Y. I. & White, J. M. (1996) J. Cell. Biol., 133:559-569.
14. Kemble, G. W., Danieli, T. & White, J. M. (1994) Cell, 76:383-391.
15. Stegmann, T., Hoekstra, D., Scherphof, G. & Wilschut, J. (1985) Biochemistry, 24:3107-3113.
16. Hoekstra, D., Klappe, K., de Boer, T. & Wilschut, J. (1985) Biochemistry, 24:4739-4745.
17. Wessels, L., Elting, M. W., Scimeca, D. & Weninger, K. (2007) Biophys. J., 93:526-538.
18. Imai, M., Mizuno, T. & Kawasaki, K. (2006) J. Biol. Chem., 281:12729-12735.
19. Elender, G., Kuhner, M. & Sackmann, E. (1996) Biosens. Bioelectron., 11:565-577.
20. Johnson, J. M., Ha, T., Chu, S. & Boxer, S. G. (2002) Biophys. J., 83:3371-3379.
21. Nollert, P., Kiefer, H. & Jahnig, F. (1995) Biophys. J., 69:1447-1455.
22. Hovis, J. S. & Boxer, S. G. (2001) Langmuir, 17:3400-3405.
23. Klonis, N., Clayton, A. H., Voss, E. W., Jr. & Sawyer, W. H. (1998) Photochem. Photobiol., 67:500-510.
24. Zimmerberg, J., Blumenthal, R., Sarkar, D., Curran, M. & Morris, S. (1994) J. Cell Biol., 127:1885-1894.
25. Chernomordik, L. V., Frolov, V. A., Leikina, E., Bronk, P. & Zimmerberg, J. (1998) J. Cell. Biol., 140:1369-1382.
26. White, J., Kartenbeck, J. & Helenius, A. (1982) EMBO J., 1:217-222.
27. Skehel, J. J., Bayley, P. M., Brown, E. B., Martin, S. R., Waterfield, M. D., White, J. M., Wilson, I. A. & Wiley, D. C. (1982) Proc. Natl. Acad. Sci. USA, 79:968-972.
28. Leikina, E., Ramos, C., Markovic, I., Zimmerberg, J. & Chernomordik, L. V. (2002) EMBO J., 21:5701-5710.
29. Mittal, A., Shangguan, T. & Bentz, J. (2002) Biophys. J., 83:2652-2666.
30. Mittal, A., Leikina, E., Chernomordik, L. V. & Bentz, J. (2003) Biophys. J., 85: 1713-1724.
31. Krumbiegel, M., Herrmann, A. & Blumenthal, R. (1994) Biophys. J., 67:2355-2360.
32. Chan D C, K. P. (1998) Cell, 89:681-684.
33. Herrler, G. & Klenk, H. D. (1987) Virology, 159:102-108.
34. Ferraretto, A., Pitto, M., Palestini, P. & Masserini, M. (1997) Biochemistry, 36:9232-9236.
35. Menke, M., Kunneke, S. & Janshoff, A. (2002) Eur. Biophys. J., 31:317-322.

It is to be understood that the embodiments of the present invention which have been described are merely illustrative of some of the applications of the principles of the present invention. Numerous modifications may be made by those skilled in the art based upon the teachings presented herein without departing from the true spirit and scope of the invention.

What is claimed is:

1. A method of monitoring virus-mediated fusion comprising:
providing a labelled, enveloped particle containing one or more viral proteins, wherein the particle has one or both of a detectably labelled envelope and a detectably labelled internal region;
providing a target membrane;
contacting the target membrane with the labelled, enveloped particle; and
monitoring fusion.

2. The method of claim 1, wherein either the envelope or the internal region has a fluorescent label.

3. The method of claim 1, wherein both the envelope and the internal region have a fluorescent label.

4. The method of claim 1, wherein the envelope and the internal region each have a different detectable label.

5. The method of claim 1, wherein the envelope contains a lipophilic, detectable label and the internal region contains a water soluble, detectable label.

6. The method of claim 4, wherein the lipophilic, detectable label is Rh110C18.

7. The method of claim 1, wherein the virus-mediated fusion is monitored for a single, enveloped particle.

8. The method of claim 1, wherein the enveloped particle is an intact virion.

9. The method of claim 5, wherein hemifusion is monitored by observing an increase or decrease in one or more photophysical properties of the lipophilic, detectable label.

10. The method of claim 9, wherein the one or more photophysical properties are selected from the group consisting of fluorescence intensity, fluorescence lifetime, emission wavelength, absorption wavelength and polarization.

11. The method of claim 9, wherein the one or more photophysical properties includes observing an instantaneous increase in brightness of the lipophilic, detectable label.

12. The method of claim 11, wherein the instantaneous increase in brightness of the lipophilic, detectable label is followed by a decrease in brightness of the of the lipophilic, detectable label.

13. The method of claim 5, wherein formation of a fusion pore is monitored by observing an increase or decrease in one or more photophysical properties of the water soluble, detectable label.

14. The method of claim 13, wherein the one or more photophysical properties are selected from the group consisting of fluorescence intensity, fluorescence lifetime, emission wavelength, absorption wavelength and polarization.

15. The method of claim 13, wherein the one or more photophysical properties includes observing a decrease in brightness of the water soluble, detectable label.

16. The method of claim 1, wherein the target membrane is selected from the group consisting of a phospholipid bilayer, a liposome, a membrane fragment and an array of bilayers.

17. The method of claim 1, wherein the target membrane is attached to a support.

18. The method of claim 17, wherein the support is selected from the group consisting of a microscope slide, a multi-well plate and a microfluidic support.

19. The method of claim 18, wherein at least a portion of the support is coated with a substance that binds phospholipids.

20. The method of claim 19, wherein the substance that binds phospholipids is dextran.

21. The method of claim 1, wherein the monitoring is performed by microscopy.

22. The method of claim 21, wherein the microscopy is fluorescence microscopy.

23. The method of claim 1, wherein the labelled, enveloped particle containing one or more viral proteins is selected from the group consisting of a virion, a virosome and a virus-like particle.

24. A method of labelling a virion comprising:
providing a virion;
providing a water soluble, detectable label; and
contacting the virion with the water soluble, detectable label to generate a labeled virion.

25. The method of claim 24, wherein the water soluble, detectable label is a fluorescent label.

26. The method of claim 25, wherein the fluorescent label is sulforhodamine B.

27. An intact virion comprising a water soluble, detectable label, wherein the water soluble, detectable label is present inside the virion.

28. A method of labelling a virion comprising:
providing a virion;
providing Rh110C18; and
contacting the virion with the Rh110C18 to generate a labeled virion.

29. A mobile lipid bilayer comprising:
a glass support, wherein at least a portion of the support is derivatized with dextran; and
a lipid bilayer attached to a least a portion of the dextran.

30. The mobile lipid bilayer of claim 29, wherein the lipid bilayer further comprises a detectable label.

31. The mobile lipid bilayer of claim 29, wherein the lipid bilayer is a phospholipid bilayer.

32. The mobile lipid bilayer of claim 23, wherein the lipid bilayer further comprises one or more viral receptors.

33. A detectable label having the structure:

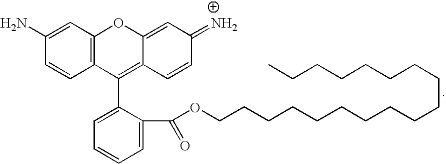

34. A method of monitoring one or more physical conditions inside a virion comprising:
providing a virion having a water soluble, detectable label inside the virion; and
observing an increase or decrease in one or more photophysical properties of the water soluble, detectable label.

35. The method of claim 34, wherein the one or more photophysical properties are selected from the group consisting of fluorescence intensity, fluorescence lifetime, emission wavelength, absorption wavelength and polarization.

36. The method of claim 34, wherein the one or more physical conditions inside a virion includes a change in pH.

37. The method of claim 36, wherein a decrease in pH is detected.

38. The method of claim 34, wherein the virion is an influenza virion.
